(19) United States
(12) Patent Application Publication
(10) Pub. No.: US 2009/0263853 A1
(43) Pub. Date: Oct. 22, 2009

(54) High-Throughput Cell-Based CFTR Assay

(75) Inventors: Matthew James Gardener, Saffron Walden (GB); Joanne Lesley Shearer, Saffron Walden (GB); David William Cronk, Saffron Walden (GB)

Correspondence Address: FOLEY & LARDNER LLP, 975 PAGE MILL ROAD, PALO ALTO, CA, 94304, US

(73) Assignee: Institute for OneWorld Health

(21) Appl. No.: 12/426,481

(22) Filed: Apr. 20, 2009

Related U.S. Application Data
(60) Provisional application No. 61/046,746, filed on Apr. 21, 2008.

(51) Int. Cl. C12Q 1/02

(52) U.S. Cl. 435/29

(57) ABSTRACT

This invention provides a high throughput screen for measuring the transport of an ion through a cystic fibrosis transmembrane conductance regulator (CFTR) of a cell that endogenously expresses CFTR. The method requires culturing the cell that endogenously expresses CFTR in the presence of an ion-sensitive compound and then contacting the cell with a CFTR activator. After a suitable amount of time after addition of the activator, the test compound is added to the cell culture medium. Thereafter, any change in the ion-sensitive compound is measured by any suitable method that can detect the change in the ion-sensitive compound, i.e. by measuring a change in the light emitted from a fluorescent compound, which can be recorded by an imaging plate reader.

### CROSS-REFERENCE TO RELATED APPLICATIONS

The application claims the benefit under 35 U.S.C. § 119(e) of provisional application U.S. Ser. No. 61/046,746, filed Apr. 21, 2008. The contents of this application are incorporated by reference into the present disclosure in its entirety.

### FIELD OF THE INVENTION

This invention relates to high-throughput cell based assays to identify compounds that modulate ion transport across the cystic fibrosis transmembrane conductance regulator (CFTR) protein.

### BACKGROUND OF THE INVENTION

Diarrhea is commonly caused by infection by a variety of bacteria, parasites and viruses and is a fundamental threat to regions lacking potable water. Preventing exposure to the pathogens responsible for diarrhea is the only way to avert infection. Unfortunately, this requires massive improvement in both sanitation and nutritional status in developing countries, which is unlikely to occur in the short term. Thus, it is a continuing threat, especially to the health of children who may lack a robust immune response. Second only to respiratory infection, diarrheal disease is responsible for approximately two million deaths in children under five years of age annually. Many who do survive have lasting health problems due to the effects of recurrent infections and malnutrition. Diarrheal diseases also are the major cause of childhood hospitalization, primarily for dehydration. Each year in developing countries, roughly four billion episodes of acute diarrhea, or approximately 3.2 episodes per child, occur among children under five years of age. See, in general, Diarrheal Diseases Fact Sheet, available from the Institute for OneWorld Health.

Diarrheal episodes can be either acute or persistent (lasting two weeks or more). Of all childhood infectious diseases, diarrheal diseases are thought to have the greatest effect on growth, by reducing appetite, altering feeding patterns, and decreasing absorption of nutrients. The number of diarrheal episodes in the first two years of life has been shown not only to affect growth but also fitness, cognitive function, and school performance.

The primary cause of death from secretory diarrhea is dehydration. As dehydration worsens, symptoms progress from thirst, restlessness, decreased skin turgor and sunken eyes to diminished consciousness, rapid and feeble pulse and low or undetectable blood pressure.

Diarrhea also often arises as a result of coinfection with other diseases such as malaria and HIV and is frequently a comorbidity factor associated with deaths due to these diseases.

It is well established that the cystic fibrosis transmembrane conductance regulator (CFTR) protein plays a pivotal role in enterotoxin-mediated secretory diarrheal disease and dehydration, which occurs as a consequence of body fluid loss following electrolyte transport across the epithelial cells lining the gastrointestinal tract. Kunzelmann and Mall (2002) Physiological Rev. 82(1):245-289. CFTR is a 1480 amino acid protein that is a member of the ATP binding cassette (ABC) transporter family. The CFTR cAMP-activated chloride (Cl⁻) channel is expressed primarily in the apical or luminal surface of epithelial cells in mammalian intestine, lungs, proximal tubules (and cortex and medulla) of kidney, pancreas, testes, sweat glands and cardiac tissue where it functions as the principal pathway for secretion of Cl(⁻)/HCO₃(⁻) and Na(+)/H(+). See Field et al. (1974) N. Engl. J. Med. 71:3299-3303 and Field et al. (1989) N. Eng. J. Med. 321:879-883.

In secretory diarrhea caused by Enterotoxigenic Escherichia coli (ETEC) and cholera, enterotoxins bind to receptors on the luminal surface of enterocytes and generate intracellular second messengers that lead to CFTR channel-opening and secretion of negatively charged ions (e.g. chloride) across the intestinal epithelia, which creates the driving force for sodium and water secretion. Kunzelmann and Mall (2002), supra. Intestinal CFTR therefore plays the central role in secretory diarrhea and the excessive loss of water, which leads to severe dehydration and rapid progression to death if untreated. Blocking ion transport across intestinal CFTR channels has been proposed as one way to treat secretory diarrhea and other disease etiologically related to ion transport across CFTR channels.

Mutations in CFTR protein, e.g., AF508, are responsible for cystic fibrosis (CF), one of the most common serious inherited diseases amongst Caucasians, affecting approximately 1 in 2,500 individuals. Pedemonte et al. (2005) J. Clin. Invest. 115(9):2564-2571. In the United States and in the majority of European countries, the incidence of carriers of the mutated CF gene is 1 in 20 to 1 in 30. CF can affect many organs including sweat glands (high sweat electrolyte with depletion in a hot environment), intestinal glands (meconium ileus), biliary tree (biliary cirrhosis), pancreas (CF patients can be pancreatic insufficient and may require enzyme supplements in the diet) and bronchial glands (chronic bronchopulmonary infection with emphysema). Hormones, such as a β-adrenergic agonist, or a toxin, such as cholera toxin, lead to an increase in cAMP, activation of cAMP-dependent protein kinase, and phosphorylation of the CFTR Cl⁻ channel, which causes the channel to open.

High throughput screening of compounds to identify CFTR modulators (activators and inhibitors) is a promising approach to discover new drugs for CFTR mediated diseases. Indeed, two classes of CFTR inhibitors (thiazolidinone and glycine hydrazide) have been identified by high-throughput screening assays. Ma et al. (2002) J. Clin. Invest. 110:1651-1658 and Muanprasat et al. (2004) J. Gen. Physiol. 124:125-137. However, to date, these assays have not provided viable therapeutic candidates due to poor water solubility and potential off-target effects. Muanprasat et al. (2007) Biol. Pharm. Bull. 30(3):502-507. Thus, a need still exists for a high throughput screen to identify active compounds that modulate CFTR. This invention satisfies this need and provides related advantages as well.

### DESCRIPTION OF THE EMBODIMENTS

High throughput screening assays for compounds which modulate CFTR ion transport described in the literature require generating stable cell lines expressing CFTR. Galietta et al. (2001) Am. J. Physiol Cell Physiol. 281:1734-1742; Jayaraman et al. (1999) Am. J. Physiol. Cell Physiol. 277:1008-1018; Pedemonte et al. (2005) J. Clin. Invest. 115(9):2564-2571; Ma et al. (2002) J. Clin. Invest. 110:1651-1658; Muanprasat et al. (2004) J. Gen. Physiol. 124:125-137 and Muanprasat et al. (2007) Biol. Pharm. Bull. 30(3):502-507. It is contemplated that the use of cells which endogenously express CFTR, particularly wild-type CFTR, for high throughput screening of new compounds which modulate CFTR ion transport will result in a robust screen to identify active compounds with the desired potency for the treatment of CFTR mediated diseases. The present invention provides robust and reliable methods for high throughput screening of new compounds and/or existing compound libraries for a new class compounds which modulate the endogenously expressed CFTR.

To that end, this invention provides a high throughput screen for compounds that modulate the transport of an ion through a cystic fibrosis transmembrane conductance regulator (CFTR) of a cell that endogenously expresses CFTR. The method requires culturing the cell that endogenously expresses CFTR in the presence of an effective amount of an ion-sensitive compound for an effective amount of time and then contacting the cell with an effective amount of CFTR activator. After a suitable amount of time after addition of the activator, the test compound is added to the culture medium. Thereafter, any change in the ion-sensitive compound is measured and correlated to the modulation (increase or decrease) of ion transport across CFTR. In one aspect, the change in the ion-sensitive compound is measured by a change in the light emitted from a fluorescent compound and is recorded by an imaging plate reader.

This invention also provides a high throughput screen for identifying a compound that inhibits transport of an ion through a cystic fibrosis transmembrane conductance regulator (CFTR) of a cell that endogenously expresses CFTR cultured in the presence of an ion-sensitive compound. The method requires contacting the cell with an effective amount of a CFTR activator and then contacting the cell with the compound. After a suitable amount of time, the change in the ion-sensitive compound is measured and correlated to the change in ion transport through the CFTR.

In an alternative embodiment, an effective amount of a potentiator compound is contacted with the cell by addition with the activator to stabilize cAMP levels.

In one aspect, the methods are performed by culturing the cell for an effective amount of time in the presence of an effective amount of FLIPR Red membrane potential dye as the ion-sensitive compound. Thereafter, an effective amount of a Forskolin (FSK) and iso-butyl-methylxanthine (IBMX) is added to the culture and the combined components are cultured for an effective amount of time. The test compound is then contacted with the cell expressing endogenous CFTR by adding it to the cell culture medium. Any change in the light emitted from the FLIPR Red membrane potential dye is measured, recorded and correlated to the change in ion transport through the CFTR of the cell.

In an alternative embodiment, an effective amount of a potentiator compound is added to the culture medium with the activator to stabilize cAMP levels.

In a yet further alternative embodiment, each of the above methods are carried out in a manner that enables the maximum inhibition value to be derived mathematically, thereby overcoming the need for a control compound or sample.

These methods are useful to identify compounds or agents that modulate, i.e., inhibit or augment, the transport of an ion across or through a CFTR and thus can identify potential therapeutics for the treatment of diseases such as diarrhea. In a further aspect, the potential therapeutic can be administered to a suitable animal model for follow on pharmacokinetic analysis or in a further aspect, to a subject such as a human patient.

### BRIEF DESCRIPTION OF THE FIGURES

Two Figures are attached to this application.

FIG. 1 shows high throughput screen results using the addition of Forskolin and IBMX before the addition of the test compounds to T84 cells loaded with FLIPR Red membrane potential dye. The black line and points represent the baseline fluorescence for each well prior to the addition of Forskolin and IBMX. The gray line and points represent fluorescence for each well following the addition of Forskolin and IBMX (F&I) then the test compound. The white line and points represent the ratio between the fluorescence of the F&I with test compound and the baseline. The X-axis represents the well number of the high throughput plate. The left Y-axis represents the relative fluorescence units (RFU) for each well, whereas the right Y-axis represents the ratio between Forskolin (Fsk) and IBMX with test compound and the baseline.

FIG. 2 shows high throughput screen results using the addition of the test compounds prior to Forskolin and IBMX to T84 cells loaded with FLIPR Red membrane potential dye. The black line and points represent the baseline fluorescence for each well prior to the addition of test compound. The gray line and points represent fluorescence for each well following the addition of the test compound then Forskolin and IBMX. The white line and points represent the ratio between the fluorescence of the test compound then Forskolin and IBMX and the baseline. The X-axis represents the well number of the high throughput plate. The left Y-axis represents the relative fluorescence units (RFU) for each well, whereas the right Y-axis represents the ratio between the test compound (Cmpd) then Forskolin and IBMX and the baseline.

### MODES FOR CARRYING OUT THE INVENTION

The practice of the present invention will employ, unless otherwise indicated, conventional techniques of tissue culture, molecular biology, chemistry and cell biology which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); Freshney (2005) Culture of Animal Cells: A Manual of Basic Technique, 5^{th} edition; Haugland, Handbook of Fluorescent Probes and Research Chemicals, 6^{th} ed. (1996); and Mayer and Walker eds. (1987) Immunochemical Methods in Cell and Molecular Biology (Academic Press, London).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

### Definitions

As used herein, certain terms may have the following defined meanings. As used in the specification and claims, the singular form “a,” “an” and “the” include plural references unless the context clearly dictates otherwise. For example, the term “a cell” includes a plurality of cells including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps.

Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated methods steps or compositions (consisting of).

By “well” it is meant generally a bounded area within a container, which may be either discrete (e.g., to provide for an isolated sample) or in communication with one or more other bounded areas (e.g., to provide for fluid communication between one or more samples in a well). For example, cells grown on a substrate are normally contained within a well that may also contain culture medium for living cells. Substrates can comprise any suitable material, such as plastic, glass, and the like. Plastic is conventionally used for maintenance and/or growth of cells in vitro.

A “multi-well vessel”, as noted above, is an example of a substrate comprising more than one well in an array. Multi-well vessels useful in the invention can be of any of a variety of standard formats (e.g., plates having 2, 4, 6, 24, 96, 384, or 1536, etc., wells), but can also be in a non-standard format (e.g., plates having 3, 5, 7, etc., wells).

A “high throughput screen” or “HTS” as used herein refers to an assay which provides for multiple candidate agents, samples or test compound to be screened simultaneously. As further described below, examples of such assays may include the use of microtiter plates that are especially convenient because a large number of assays can be carried out simultaneously, using small amounts of reagents and samples. The methods are easily carried out in a multiwell format including, but not limited to, 96-well and 384-well formats and automated.

“Ion transport” is the movement of ions across a cell membrane by either an active or passive mechanism. For example, active transport comprises the hydrolysis of ATP by the transmembrane protein, whereas passive transport does not. For the purpose of this invention, the ion is transported across CFTR and is at least one halide which includes, for example one or more of iodide (I⁻), chloride (Cl⁻) and bromide (Br⁻). In one aspect, the ion is a chloride ion.

“Cystic fibrosis transmembrane conductance regulator” or “CFTR” functions as a chloride channel and controls the regulation of other transport pathways. CFTR and outwardly rectifying chloride channels (ORCCs) are distinct channels but are linked functionally via an unknown regulatory mechanism. The CFTR gene has been mapped to the chromosome 7q31.2. Riordan et al. (1989) Science 245:1066-1073. The predicted protein has 1,480 amino acids with a molecular mass of 168,138 Daltons.

A “subject,” “individual” or “patient” is used interchangeably herein, and refers to a vertebrate, preferably a mammal, more preferably a human. Mammals include, but are not limited to, murines, rats, rabbits, simians, bovines, ovines, porcines, canines, felines, farm animals, sport animals, pets, equines, and primates, particularly humans.

“Cell” or “host cell” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. It should be understood, without explicit repeat recitation, that the cells may be of any animal type, including without limitation, human, bovine, porcine and/or simian. Cells that endogenously express CFTR include, for example, epithelial cells of the lung, liver, pancreas, digestive tract, reproductive tract and skin. Such cells are typically involved in the production of mucus, sweat, saliva, tears or the digestive enzymes. In one aspect, the cells that endogenously express CFTR have been immortalized for convenient use in culturing and long-term survival and replication. Non-limiting examples of cultured cell lines that endogenously express CFTR are the T84 or HT29 cell lines that are commercially available from the American Type Culture Collection (ATCC), Rockville Md., USA. Other cell lines that endogenously express CFTR include, but are not limited to, CFPAC-1, DSL-6A/C1, IB3-1, S9, C38 and Capan-1 which are commercially available from the American Type Culture Collection (ATCC). Furthermore, the European Collection of Cell Cultures (ECACC) also provide cell lines that endogenously express CFTR, e.g., CACO-2, CFPAC-1 and HT29 gluc C1. In one aspect, the cell that endogenously expresses CFTR is a primary epithelial cell or cell line taken directly from a living organism or subject, but which has not been immortalized.

“Endogenous” refers to a substance that originates from within an organism, tissue or cell. In one embodiment, the substance is a peptide, polypeptide or protein. In another embodiment, term “endogenous” excludes a peptide, polypeptide or protein that is expressed by recombinant expression systems or exogenously provided by methods known to one of skill in the art. In another embodiment, the expression of an endogenous polypeptide or protein may be up regulated or down regulated by methods known to one of skill in the art, which enhance or inhibit expression.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. The growth of explants taken directly form the living organism (e.g. biopsy material) is also known as primary cell culture.

An “ion-sensitive compound” refers to compound which is capable of being detected by the ionic strength or concentration of ions in the surrounding media. The concentration of ions or a change in the concentration of ions can be measured by a change in florescence by the ion-sensitive compound. Examples of such compounds are provided herein.

A “CFTR activator” refers to a chemical or biological compound which initiates the transport of chloride ions through the cAMP regulated channel of CFTR. Non-limiting examples of CFTR activators are Forskolin (FSK), cholera toxin (CTX), enterotoxin from Escherichia coli (ETEC), Isoproterenol, protein kinase A (PKA), genistein, apigenin, 8-cyclopentyl-1,3-dipropylxanthine (CPX) and 8-methoxypsoralen (8-MPO). The actions of some or all of these agents may be potentiated by agents that stabilize cAMP levels; non-limiting examples of these include iso-butyl-methylxanthine (IBMX) and milrinone. All of these agents are available for purchase from Sigma-Aldrich, Inc. The mechanism by which the activator initiates the transport of the halide, e.g., chloride ions, can vary depending on the compound. It is understood that one of skill in the art can readily determine the appropriate compound to use in the herein described assay.

The term “test compound” refers to a chemical or agent, such as a biological agent such as an antibody, peptide, protein or nucleic acid, to be tested by one or more screening method(s) of the invention as a putative modulator of the target. A test compound can be any chemical, such as an inorganic chemical or an organic chemical. Usually, various predetermined concentrations of test compounds are used for screening, such as 0.01 micromolar, 1.0 micromolar and 10.0 micromolar and various intervening amounts. Test compound controls can include the measurement of a signal in the absence of the test compound or comparison to a compound known to modulate the target.

The term “fluorescent” refers to any substance or agent that is capable of exhibiting “fluorescence” (or “photoluminescence”), which is the emission of light triggered by the molecular absorption of a photon with shorter wavelength. Fluorescence thus is dependent on an “excitation light source” that is distinct from the longer wavelength fluorescence “emission” emanating from the fluorophore. Detection of fluorescence emission requires that a detector that responds only to the emission light and not to the excitation light.

In the field of compound screening, “fluorescence imaging plate readers” are typically used in cell-based assays, which are run on collections of cells. The measured response is usually an average over the cell population. For example, a popular instrument used for ion channel assays is disclosed in U.S. Pat. No. 5,355,215. A typical assay consists of measuring the time-dependence of the fluorescence of an ion-sensitive dye, the fluorescence being a measure of the intra-cellular concentration of the ion of interest which changes as a consequence of the addition of a chemical compound.

As is known to those of skill in the art, a response is detected when there is a change in a property of the fluorescence light, such as a change in the intensity, polarization, energy transfer, lifetime, and/or excitation or emission wavelength distribution. The detectable response may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, and/or other property.

### High Throughput Screening Methods

As noted above and explained in more detail herein, this invention provides a high throughput screen to assay and measure transport of an ion through a cystic fibrosis transmembrane conductance regulator (CFTR) of a cell that endogenously expresses CFTR. The method requires culturing the cell that endogenously expresses CFTR in the presence of an effective amount of an ion-sensitive compound for an effective amount of time and then contacting the cell with an effective amount of CFTR activator.

In one aspect, the methods described herein include identifying and obtaining a cell that endogenously express CFTR. Cells that endogenously express CFTR can be obtained from commercial sources such as the American Type Culture Collection (ATCC). In one aspect, to practice the methods described herein, said cells are cultured under appropriate conditions for conducting the assays described herein such as, but not limited to, seeding and/or growing the cells to a predetermined cell density, growing the cells in the presence of growth media or seeding and/or growing the cells in a tissue culture plate suitable for measuring fluorescence. Prior to conducting the assay, the cells are washed, appropriate assay media is added and the cells are then contacted with an effective amount of an ion-sensitive dye and incubated for a sufficient amount of time to allow incorporation of the ion-sensitive compound into the cytoplasm of the cells. The assay begins by contacting the cells with an effective amount of a CFTR activator followed by the addition of a test compound. The ability of the test compound to inhibit the transport of an ion through the CFTR in measured by detecting a change in the ion-sensitive dye such as, but not limited to, a change in fluorescence. In this aspect, a decrease in fluorescence identifies that test compound as an inhibitor of ion transport through the CFTR.

In one aspect, the cell is a primary cell isolated from a subject or patient such as a mammal, e.g., a human cell, a bovine cell or a simian cell, that endogenously expresses CFTR. CFTR is expressed primarily in the apical or luminal surface of epithelial cells in mammalian intestine, lungs, proximal tubules (and cortex and medulla) of kidney, pancreas, testes, sweat glands and cardiac tissue. Primary cells that express CFTR include but are not limited to epithelial cells of the lung, liver, pancreas, digestive tract, reproductive tract and skin. If primary cells are used, they are cultured under conditions that allow for cell proliferation, uptake of ion-sensitive compounds or any condition required for the analysis.

In another aspect, the cell is a cultured cell that endogenously expresses CFTR and are not limited to epithelial cells. Examples of such include but are not limited to a T84 cell and a HT29 cell each of which is commercially available from the American Type Culture Collection (ATCC).

Typically, approximately 25,000 cells are plated at a concentration of about 1000 to about 2000 cells per μl of growth medium. The cells are allowed to settle and grow but not reach confluence. For the purpose of illustration only, the cells shall grow to about 50%, or alternatively 60%, or alternatively 70%, or yet further up to 80% confluence.

An effective amount of the ion-sensitive compound is added to the cell culture after the cells have reached the desired confluence and the cells are then allowed to incubate with the ion-sensitive compound to allow incorporation of the ion-sensitive compound into the cytoplasm of the cell. Typically, for cells cultured in microtiter plates, the amount of assay buffer is 100 μl per well for 96-well plates or 25 μl for 384-well plates. The exact amount will differ with the cell and ion-sensitive compound but can be empirically determined by one of skill in the art. Typical incubation ranges include from about 10 to about 90 minutes. Examples of ion-sensitive compounds useful in the methods described herein include voltage sensitive dyes. Voltage sensitive dyes have been used to evaluate cellular membrane potential. Zochowski et al. (2000) Biol. Bull. 198:1-21. As used herein, membrane potential dyes or voltage-sensitive dyes refer to molecules or combinations of molecules that enter depolarized cells, bind to intracellular proteins or membranes and exhibit enhanced fluorescence. Voltage-sensitive dyes include, but are not limited to, modified bisoxonol dyes, sodium dyes, potassium dyes and thorium dyes. The dyes enter cells and bind to intracellular proteins or membranes, therein exhibiting enhanced fluorescence and red spectral shifts. Epps et al. (1994) Chem. Phys. Lipids 69:137-150. Increased depolarization results in more influx of the anionic dye and thus an increase in fluorescence.

Other examples of such compounds include FLIPR Red membrane potential dye and FLIPR Blue membrane potential dye, which are available commercially from Molecular Devices, a part of MDS Analytical Technologies. Patents that describe ion-sensitive compounds include U.S. Pat. Nos. 6,420,183; 5,641,684; 5,550,268; 5,608,059; 5,541,330 and international publication WO 1993/012428. In one embodiment, the ion-sensitive dyes are FMP dyes available from Molecular Devices (Catalog Nos. R8034, R8123). Other suitable dyes could include dual wavelength FRET-based dyes such as DiSBAC2, DiSBAC3, and CC-2-DMPE (Invitrogen Cat. No. K1016). Chemical Name: Pacific Blue™ 1,2-ditetradecanoyl-sn-glycero-3-phosphoethanolamine, triethylammonium salt.

The ion-sensitive compounds are incorporated into the cell membrane by contacting the cells with a solution comprising a membrane-permeable derivative of the dye. However, the loading process may be facilitated where a more hydrophobic form of the dye is used.

The cells endogenously expressing CFTR of the assay are preloaded with the fluorescent dyes for about 30 to about 240 minutes prior to addition of candidate compounds. Preloading refers to the addition of the ion-sensitive compound for a period prior to candidate compound addition during which the dye enters the cell and binds to intracellular lipophilic moieties. Cells are typically treated with about 1 to about 10 μM buffered solutions of the dye for about 20 to about 60 minutes at 37° C. In some cases it is necessary to remove the dye solutions from the cells and add fresh assay buffer before proceeding with the assay.

After incubation with the ion-sensitive compound, an effective amount of an activator is added to the culture medium. The term “activator” intends a chemical or biological compound which initiates the transport of ions through the cAMP regulated channel of CFTR, examples of which include but are not limited to at least one of Forskolin (FSK), cholera toxin (CTX), enterotoxin from Eschericia coli (ETEC), Isoproterenol, protein kinase A (PKA), genistein, apigenin, 8-cyclopentyl-1,3-dipropylxanthine (CPX), and 8-methoxypsoralen (8-MPO). The actions of some or all of these agents may be “potentiated” by the co-addition of agents that stabilize cAMP levels. As used herein, “co-addition” intends not only simultaneous addition but also after the ion-sensitive dye and prior to the additional of the test compound. Non-limiting examples of potentializing agents include, but are not limited to iso-butyl-methylxanthine (IBMX) and milrinone. The activator, alone or in combination with the potentiator, is added to a final concentration of about 1 μM to about 100 mM, or alternatively, about 10 μM to about 10 mM, or alternatively, about 100 μM to 1 mM. The exact amount will differ with the cell and ion-sensitive compound used but can be empirically determined by one of skill in the art. In a separate embodiment, the activator is one or more of an effective amount of Forskolin (FSK) or Isoproterenol and an effective amount of IBMX or milrinone as the potentiator is added to the culture medium, as described above.

After a suitable amount of time after addition of the activator, with or without a potentiator, which includes from about 10 seconds to about 5 minutes, or alternatively about 20 seconds to about 4 minutes, or alternatively about 30 second to 3 minutes, or alternatively, about 40 seconds to about 2 minutes, or alternatively about 50 seconds to about 90 seconds, the test compound or agent is added to the culture medium so that it is contacted with the cell endogenously expressing CFTR. Thereafter, any change in the ion-sensitive compound is measured by any suitable method and correlated to the measurement of ion transport across CFTR. Modulation in fluorescence can be monitored every second over a period of about 5 to 10 minutes.

Typically, the monitoring of fluorescence is reduced following the first minute to a slower sampling rate of about every 10 seconds until the completion of the experiment. In one aspect, the change in the ion-sensitive compound is measured by a change in the light emitted from a fluorescent compound and recorded by an imaging plate reader. A reduction in the fluorescence emitted from the ion-sensitive compound indicates that the compound or agent is an inhibitor and a increase indicates that the compound or agent facilitates ion transport. Because chloride, bromide and iodide ions are transported across endogenous CFTR, the assay can be used to assay for transport of these ions. In a particular embodiment, chloride ion transport is measured. Additionally, fluid will follow the transport of ions across CFTR, compounds or agents that inhibit ion transport are candidate anti-diarrhea agents.

Although positive and negative controls (with a known inhibitor or facilitator) can be run simultaneously with the test compound, one advantage of the present method is that a negative control is not necessary. In the current invention, the lack of a suitably robust negative control, i.e. a facilitator, is overcome by mathematical derivation of the maximum inhibition value for the assay using the data captured during the course of the experiment. This mathematical derivation adds to the robustness and reliability of the assay. However, in one aspect, a positive and/or a negative control is run simultaneously with the test agent. In another aspect, only a positive control is run.

After addition of the test compound, the change in the ion-sensitive compound (as a measure of ion flux) is measured by methods that are appropriate for the particular compound. For example, optical methods are particularly suitable methods for high throughput screening of fluorescent detection. Optical methods permit measurement of the entire course of ion flux in a single cell as well as in groups of cells. Eidelman, O. et al., (1989) Biophys. Acta 988:319-334. Present day optical readers detect fluorescence from multiple samples in a short time and can be automated. Fluorescence readouts are used widely both to monitor intracellular ion concentrations and to measure membrane potentials.

The methods and systems of this invention can rapidly screen sets of compounds, such as, individual compound libraries, compound mixtures, chemical libraries, and the like. Individual compounds from a set of compounds can be screened individually one at a time. Also, groups of compounds or compound mixtures can be screened to quickly narrow a larger number down to a more manageable size. The smaller group can be further screened for individual compounds or mixtures of compounds. One of skill in the art can easily recognize additional variations that are appropriate for a given set of circumstances.

Detecting and recording alterations in the spectral characteristics of the dye in response to changes in membrane potential may be performed by any means known to those skilled in the art. As used herein, a “recording” refers to collecting and/or storing data obtained from processed fluorescent signals, such as are obtained in fluorescent imaging analysis.

In some embodiments, the assays of the present invention are performed on isolated cells using microscopic imaging to detect changes in spectral (i.e., fluorescent) properties. In other embodiments, the assay is performed in a multi-well format and spectral characteristics are determined using a microplate reader.

A suitable configuration for single cell imaging involves the use of a microscope equipped with a computer system. One example of such a configuration, ATTO's Attofluor™ RatioVision™ real-time digital fluorescence analyzer from Carl Zeiss, is a completely integrated work station for the analysis of fluorescent probes in living cells and prepared specimens (ATTO, Rockville, Md.). The system can observe ions either individually or simultaneously in combinations limited only by the optical properties of the probes in use. The standard imaging system is capable of performing multiple dye experiments such as FMP (for sodium) combined with GFP (for transfection) in the same cells over the same period of time. Ratio images and graphical data from multiple dyes are displayed online.

When the assays of the invention are performed in a multi-well format, a suitable device for detecting changes in spectral qualities of the dyes used is a multi-well microplate reader. Suitable devices are commercially available, for example, from Molecular Devices (FLEXstation™ microplate reader and fluid transfer system or FLIPR™ system), from Hamamatsu (FDSS 6000), PerkinElmer Life and Analytical Sciences (CellLux™) and the “VIPR” voltage ion probe reader (Aurora, Bioscience Corp. Calif., USA). The FLIPR-Tetra™ is a second generation reader that provides real-time kinetic cell-based assays using up to 1536 simultaneous liquid transfer systems. All of these systems can be used with commercially available dyes such as FMP, which excites in the visible wavelength range.

Using the FLIPR™ system (commercially available from Molecular Devices), the change in fluorescent intensity is monitored over time and can be graphically displayed. For example, the addition of an activating composition causes an increase in fluorescence, while the addition of a compound that blocks or inhibits CFTR activation blocks this increase.

Several commercial fluorescence detectors are available that can inject liquid into a single well or simultaneously into multiple wells. These include, but are not limited to, the Molecular Devices FlexStation (eight or sixteen wells), BMG NovoStar (two wells) and Aurora VIPR (eight or thirty-two wells). Typically, these instruments require 12 to 96 minutes to read a 96-well plate in flash luminescence or fluorescence mode (1 min/well). An alternative method is to inject the modulator into all sample wells at the same time and measure the luminescence in the whole plate by imaging with a charge-coupled device (CCD) camera, similar to the way that calcium responses are read by calcium-sensitive fluorescent dyes in the FLIPR®, FLIPR-384 or FLIPR-Tetra instruments. Other fluorescence imaging systems with integrated liquid handling are expected from other commercial suppliers such as the Perkin Elmer CellLux—Cellular Fluorescence Workstation and the Hamamatsu FDSS6000 System. These instruments can generally be configured to proper excitation and emission settings to read FMP dye (535ₑₓ±15 nm, 565ₑₘ±15 nm). The excitation/emission characteristics differ for each dye, therefore, the instruments are configured to detect the dye chosen for each assay. Selection of appropriate dye and configuration of the instruments to detect the dye chosen for each experiment is within the skill of the artisan practicing the experiments. Additionally, the use of specialized optical filters can be used to improve detection of the light emitted from the ion-sensitive compound or reduce background from the cells or media used in the assay. The type and properties of the filter will depend on the ion-sensitive compound used and can be empirically determined by one of skill in the art.

After a potential therapeutic agent is identified, pharmacokinetic profiling can by pursued by administration of an amount of the agent to an animal host as a suitable animal model. After further efficacy and safety analysis, the drug can be administered to a human patient to treat or ameliorate the symptoms of disease mediated by CFTR ion transport such as diarrhea.

### Candidate Compounds

The assays are designed to screen large chemical libraries by automating the assay steps, and the compounds are provided from any convenient source to the cells. Assays can be run in parallel (e.g., in microtiter formats on microtiter plates in robotic assays with different candidate compounds in different wells on the same plate).

As noted above, candidate compounds employed in the screening methods of this invention include without limitation, synthetic organic compounds, naturally occurring products, e.g., polypeptides and nucleic acids. Essentially any chemical compound can be screened as a potential modulator in the assays of the invention. They can be synthesized de novo or purchased from a supplier such as ChemDiv (San Diego, Calif.), Sigma-Aldrich (St. Louis, Mo.) and Fluka Chemika-Biochemica-Analytika (Buchs Switzerland).

The screening methods of this invention are particularly suited to screen small organic molecule or peptide library containing a large number of potential CFTR activators or inhibitors. Such “chemical libraries” are then screened in one or more assays, as described herein, to identify those library members (particular chemical species or subclasses) that display a desired characteristic activity. The compounds thus identified can serve as conventional “active compounds” or can themselves be further modified by one skilled in the art.

The patent and technical literature provide numerous technical methodologies for the preparation of compound libraries. (See, e.g., U.S. Pat. No. 5,010,175; Furka (1991) Int. J. Pept. Prot. Res. 37:487-493; Houghton et al. (1991)Nature 354:84-88; PCT Publication No. WO 91/19735; PCT Publication No. WO 93/20242; PCT Publication No. WO 92/00091; U.S. Pat. No. 5,288,514; Hobbs et al. (1993) Proc. Nat. Acad. Sci. USA 90:6909-6913; Hagihara et al. (1992) J. Amer. Chem. Soc. 114:6568; Hirschmann et al. (1992) J. Amer. Chem. Soc. 114:9217-9218; Chen et al. (1994) J. Amer. Chem. Soc. 116:2661; Cho et al. (1993) Science 261:1303; Campbell et al. (1994) J. Org. Chem. 59:658; U.S. Pat. No. 5,539,083; Vaughn et al. (1996) Nature Biotechnology 14:309-314; PCT/US96/10287; Liang et al. (1996) Science 274:1520-1522 and U.S. Pat. No. 5,593,853.)

Devices for the preparation of combinatorial libraries are commercially available (see, e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.).

This invention also provides a kit for the practice of the methods of this invention that includes, without limitation the reagents necessary to conduct the screen(s) and instructions for their use and interpretation of the data. The kit may optionally include a variety of other reagents. Such reagents include, but are not limited to, salts, solvents, neutral proteins, e.g. albumin, detergents, etc., which may be used to facilitate optimal protein-protein binding and/or to reduce non-specific or background interactions. Examples of solvents include, but are not limited to, dimethyl sulfoxide (DMSO), ethanol and acetone, and are generally used at a concentration of less than or equal to 1% (v/v) of the total assay volume. In addition, reagents that otherwise improve the efficiency of the assay, such as protease inhibitors, anti-microbial agents, etc. may be used. Further, the mixture of components in the method may be added in any order that provides for the requisite binding. The compounds identified using the disclosed assay are potentially useful as treatments for CFTR mediated diseases. The amount of such a modulating compound will be an effective amount that yields the desired degree of improved clinical or non-clinical parameters.

### EXPERIMENTAL EXAMPLES

In these examples and elsewhere, abbreviations have the following meanings:
μL or μl=Microliter
nL or nl=Nanoliter
g=Gram
h=Hour
M=Molar
mg=Milligram
min=Minute
mL=Milliliter
mM=Millimolar
μM=Micromolar
mm=Millimeter
mmol=Millimoles
mol=Moles
cm=Centimeter
nm=Nanometer
PBS=Phosphate buffered saline
HEPES=4-(2-hydroxyethyl)-1-piperazineethanesulfonic acid
DMEM:F12=Dulbecco's Modification of Eagle's Medium:Ham's F-12 50:50 Mixture
FSK=Forskolin
IBMX=Iso-butyl-methylxanthine
DMSO=Dimethyl sulfoxide

### Example 1

### High Throughput Screen for Inhibitory Compounds Using T84 Cells

### Cell Culture and Plating

0.6×10⁶ T84 cells are seeded into T-175 flasks and cultured for 4 days or until they reach >70% coverage. The cells are then detached using trypsin, washed with PBS, counted and resuspended in growth media to give a concentration of 1.25×10⁶/ml. The cells are then plated at a density of 25,000 in 20 μl into black sided clear bottom tissue culture treated plates and left overnight at 37° C.

Prior to the start of the assay, the growth media is replaced with serum free assay media. To facilitate this, cells are washed twice with approximately 70 μl of assay buffer, the plate is then “tapped” dry and 20 μl of DMEM:F12 (Gibco 31331) containing 20 mM HEPES is added back. The cell plates are then returned to the incubator (37° C., 5% CO₂) for 15 minutes before membrane potential sensitive dye loading.

### Preparation of Assay Buffer

The assay buffer is HBSS (with Ca²⁺/Mg²⁺) with 20 mM HEPES, pH7.38. To prepare the buffer, with continual stirring, 2.38 g HEPES (Sigma H3375) are dissolved into 500 ml HBSS preheated to 35° C. Volumes are scaled up as required. Using a pre-calibrated pH meter, the pH is adjusted to approximately 7.38 with 10M NaOH. 7.38 to 7.41 is an acceptable range. The assay buffer is then returned to a 35° C. water bath for the duration of assay; an aliquot is taken and cooled to room temperature for the preparation of FSK/IBMX and compounds.

### Preparation of Forskolin (Fsk) and Ibmx

To prepare FSK and IBMX, dilute 10 mM FSK and 100 mM IBMX stocks (aliquoted and stored at −20° C.) in assay buffer to give a solution containing 100 μM FSK and 1 mM IBMX. This then undergoes a further 10-fold dilution in the assay and is equivalent to 10 μM FSK and 100 μM IBMX on the cells. For each 10 ml assay buffer 100 μl of 10 mM FSK and 100 μl of 10 mM IBMX is required. 5 μl is needed per well/experiment, volumes are scaled up accordingly. The working solution is prepared on the day of the assay and stored at 4° C. Just prior to running the assay the stimulation plate is prepared by adding FSK/IBMX to columns 1-23 and buffer to column 24.

### Preparation of Test Compounds

In a 384 well plate, 10 mM stock compound is diluted 20-fold in assay buffer to give 500 μM (e.g. usually 2 μl of compound diluted with 38 μl buffer). 6 μl are then stamped into a separate plate and further diluted 10-fold by the addition of 54 μl assay buffer. 12.5 μl is then added in the assay (see later) i.e. compound is prepared in the assay compound plate at 5× the final concentration.

### Assay Procedure (HTS of Library Compounds)

1. Cell plates are washed twice with assay buffer, tapped dry and 20 μl serum free assay media added to each well. The plates are then returned to a 37° C. incubator for 15 minutes.
2. 25 μl of Red membrane potential dye (Molecular devices, R8126; 1 vial reconstituted in 100 ml assay buffer) is added to each well and incubated for a further 45 minutes at 37° C.
3. The cell plate is then transferred to the FLIPR and fluorescence measured with filter 2 in place. Whilst recording every second channel stimulation is achieved by the addition of 5 μl of 100 μM FSK and 1 mM IBMX. After 50 seconds, data capture is slowed to 10 second sampling for a further 220 seconds. Test compounds are then tested for their ability to inhibit the channel by addition of 12.5 μl from a pipettor height of 50 μl (dispense speed 20 μl/sec). Decreases in fluorescence are then monitored over a 5 minute period, with measurements taken every second for the first minute followed by 10 second sampling until the end of the experiment.

### Data Handling for HTS

The data is exported as a time sequence file and passed through an automated script to extract the pre-stimulation fluorescence value (termed “X”), post stimulation fluorescence value (termed “Y”) and the endpoint post compound fluorescence value (termed, “Z”). Any plate effects are then corrected out using the runwise multiplicative correction factor function in Genedata and the corrected output is then used in XLfit to calculate the % Inhibition for each well. The equation below is used to calculate % Inhibition:

%I=100*(1−((z/y^{cmpd}−x/y^{cntl}/(z/y^{cntrl}−x/y^{cntrl})))

Positive control wells are those which received 5 μl FSK/IBMX followed by 12.5 μl buffer/DMSO; negative control wells are those which received 5 μl buffer/2% DMSO followed by 12.5 μl buffer/2.5% DMSO.

### Results

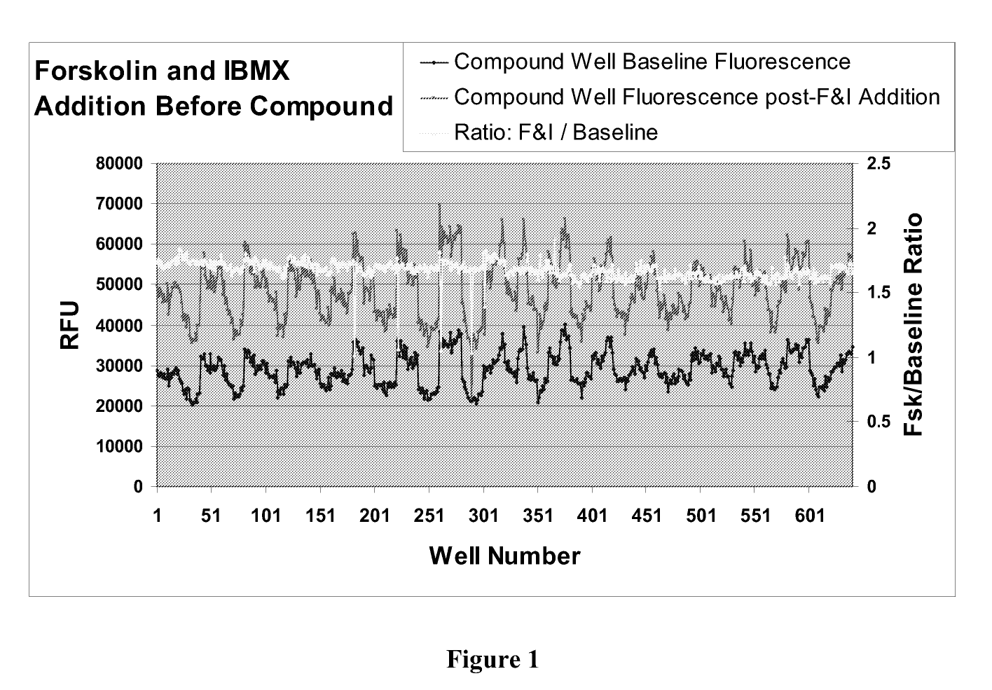

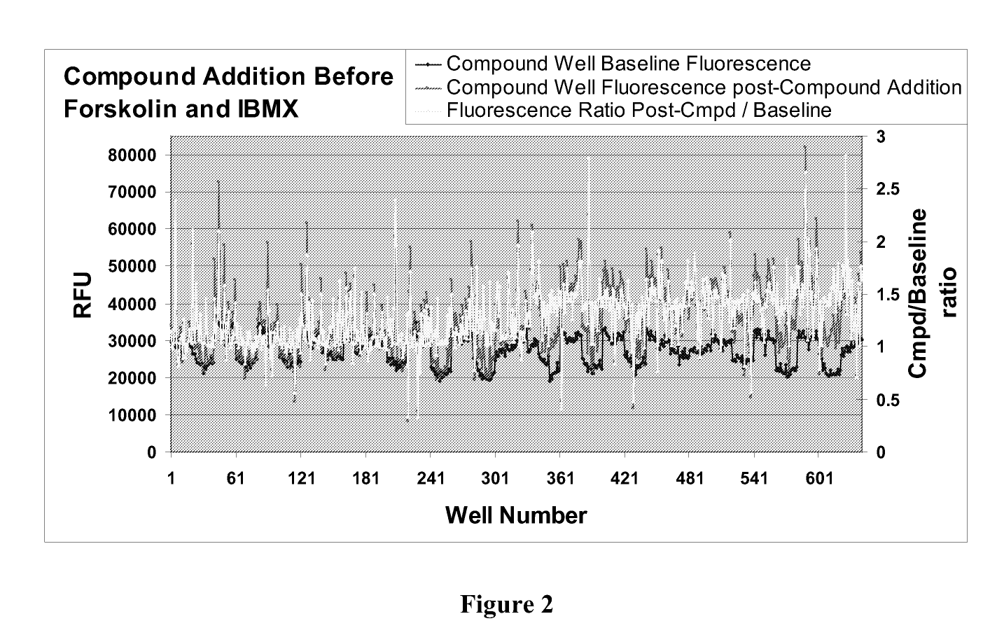

The effect of the addition of Forskoin and IBMX before the addition of the test compounds in the HTS assay when expressed as a ratio shows the variability is significantly reduced to give a robust and reliable value (FIG. 1). Test compounds which inhibit CFTR ion transport show a low ratio compared to the baseline. However, when the addition of Forskoin and IBMX and the test compound are reversed, the baseline fluorescence values measured are very similar to FIG. 1, but test compound wells elicits a small inconsistent change in the measured fluorescence and therefore the variability is markedly increased (FIG. 2). This cannot be corrected by expressing the data in ratio form compared to baseline, thus rendering the assay insufficiently robust for screening purposes in this configuration.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A high throughput screen for measuring the transport of an ion through a cystic fibrosis transmembrane conductance regulator (CFTR) of a cell that endogenously expresses CFTR cultured in the presence of an ion-sensitive compound, comprising:
a. contacting the cell with a CFTR activator;
b. contacting the cell with a test compound; and
c. measuring a change in the ion-sensitive compound, thereby measuring the ion transport through the CFTR of the cell.

2. The high throughput screen of claim 1, wherein the cell is a T84 cell or a HT29 cell.

3. The high throughput screen of claim 1, wherein the ion-sensitive compound is a voltage-sensitive dye.

4. The high throughput screen of claim 3, wherein the voltage-sensitive dye is FLIPR Red membrane potential dye.

5. The high throughput screen of claim 1, wherein the ion is chloride, bromide or iodide.

6. The high throughput screen of claim 1, wherein the CFTR activator comprises one or more compound selected from the group consisting of Forskolin (FSK), cholera toxin (CTX), enterotoxin from Escherichia coli (ETEC), Isoproterenol, protein kinase A (PKA), genistein, apigenin, 8-cyclopentyl-1,3-dipropylxanthine (CPX) and 8-methoxypsoralen (8-MPO).

7. The high throughput screen of claim 1, further comprising contacting the cell with a potentiator and the CFTR activator.

8. The high throughput screen of claim 1, wherein the change in the ion-sensitive compound is measured by a fluorescence imaging plate reader or an equivalent thereof.

9. A high throughput screen for measuring the modulation of the transport of an ionthrough a cystic fibrosis transmembrane conductance regulator (CFTR) of a cell that endogenously expresses CFTR cultured in the presence of a FLIPR Red membrane potential dye, comprising the steps of:
a. contacting the cell with Forskolin (FSK) and iso-butyl-methylxanthine (IBMX);
b. contacting the cell with a test compound; and
c. measuring a change in the FLIPR Red membrane potential dye, thereby measuring the modulation of the transport of an ion through the CFTR of the cell.

10. A high throughput screen for identifying a compound that inhibits transport of an ion through a cystic fibrosis transmembrane conductance regulator (CFTR) of a cell that endogenously expresses CFTR cultured in the presence of an ion-sensitive compound, comprising:
a. contacting the cell with a CFTR activator;
b. contacting the cell with the compound; and
c. measuring a change in the ion-sensitive compound, wherein a reduction in the transport of an ion identifies the compound as one that inhibits ion transport through CFTR.

11. The high throughput screen of claim 10, wherein the cell is a T84 cell or a HT29 cell.

12. The high throughput screen of claim 10, wherein the ion-sensitive compound is a voltage-sensitive dye.

13. The high throughput screen of claim 12, wherein the voltage-sensitive dye is FLIPR Red membrane potential dye.

14. The high throughput screen of claim 10, wherein the ion is chloride, bromide or iodide.

15. The high throughput screen of claim 10, wherein the activator comprises one or more compound selected from the group consisting of Forskolin (FSK), cholera toxin (CTX), enterotoxin from Escherichia coli (ETEC), Isoproterenol, protein kinase A (PKA), genistein, apigenin, 8-cyclopentyl-1,3-dipropylxanthine (CPX) and 8-methoxypsoralen (8-MPO).

16. The high throughput screen of claim 10, further comprising contacting the cell with a potentiator and the activator.

17. The high throughput screen of claim 10, wherein the ion-sensitive compound is a voltage-sensitive dye and the change in the ion-sensitive compound is recorded by a fluorescence imaging plate reader or an equivalent thereof.

18. A high throughput screen for identifying a compound that inhibits the transport of an ion through a cystic fibrosis transmembrane conductance regulator (CFTR) of a cell that endogenously expresses CFTR cultured in the presence of a FLIPR Red membrane potential dye, comprising the steps of:
a. contacting the cell with an effective amount of Forskolin (FSK) and iso-butyl-methylxanthine (IBMX);
b. contacting the cell with the compound; and
c. measuring a change in the FLIPR Red membrane potential dye, wherein a decrease in fluorescence identifies the compound as an inhibitor of ion transport.
